(19) United States
(12) Patent Application Publication
(10) Pub. No.: US 2008/0050350 A1
(43) Pub. Date: Feb. 28, 2008

(54) Method for introducing an exogenous gene into a stem cell

(75) Inventors: Jenn-Rong YANG, Tainan (TW); Lih-Ren CHEN, Tainan (TW); Yow-Ling SHIUE, Tainan (TW); Shinn-Zong Lin, Tainan (TW); Chia-Hsin LIAO, Tainan (TW)

Correspondence Address: LADAS & PARRY, 26 WEST 61ST STREET, NEW YORK, NY, 10023, US

(73) Assignee: LIVESTOCK RESEARCH INSTITUTE COUNCIL OF AGRICULTURE EXECUTIVE YUAN

(21) Appl. No.: 11/715,699

(22) Filed: Mar. 8, 2007

(30) Foreign Application Priority Data
Aug. 25, 2006 (TW) 095131416

(51) Int. Cl. C12N 15/87; C12N 5/06; G01N 33/00; G09B 25/08

(52) U.S. Cl. 424/93.21; 435/326; 435/461; 800/3

(57) ABSTRACT

The subject invention relates to a method for introducing an exogenous gene into a stem cell, which comprises introducing the exogenous gene into the stem cell under specific electroporation conditions. The invention also relates to a stem cell stably expressing an exogenous gene, a transgenic animal and a pharmaceutical composition.

### BACKGROUND OF THE INVENTION

1. Field of the Invention

The invention relates to gene introduction. Particularly, the invention relates to a method for introducing an exogenous gene into a stem cell.

2. Description of the Related Art

Mammalian stem cells have the property of prolongated proliferation and are able to differentiate into adult cells that have specific morphology and to physiological functions. Having the ability to further differentiate into specific adult cells and its plasticity, stem cells are excellent materials for exploring embryonic development, and can be induced to differentiate into cells of three germ layers for the manufacture of adult cells under proper conditions. Such adult cells are utilized in repairing, regenerating or recovering adult cells, tissue and even organs suffered from mechanical, chemical or biological damages and diseases. Therefore, the stem cells can be used in regenerative medicine, oncology and therapeutic cloning. For example, human embryonic stem cells are proven to be able to express receptors of neuronal cells (Reubinoff et al, 2001, Nat. Biotechnol. 19:1134-1140; Schuldiner et al, 2001, Brain Res. 913:201-205). Adult stem cells are also reported to be isolated from the dermis of mice. Because of the plasticity of adult stem cells, precursor cells of specific adult cells are further derived from such adult stem cells for use in adult stem cell transplantation (Shufaro and Reubinoff, 2004, Best Pract Res Clin Obstet Gynaecol. 18(6): 909-927).

In the field of stem cell study, introduction of an exogenous gene is an important technique. For example, the growth and differentiation of a stem cell can be detected by introducing a reporter gene. In gene therapy, introducing an exogenous gene is also a key step. Several processes of inducing an exogenous gene have been reported, such as introducing an exogenous gene into a stem cell with the assistance of a retrovirus, or introducing an exogenous gene by using a viral vector of an adenovirus or lentivirus (Pfeifer et al., 2002, Proc. Natl. Acad. Sci. USA. 99:2140-2145; Smith-Arica et al., 2003, Cloning Stem Cells. 5:51-62). However, retroviruses are subjected to epigenetic modification and the retroviral expression is short (Chan et al., 1998, Proc. Natl Acad. Sci. USA 95:14028-14033). Furthermore, the application of lentiviral vector transfection to higher mammals is still under question (Wolfgang et al., 2001, Proc. Natl. Acad. Sci. USA 98:10728-10732).

Liposome, such as Lipofectamine™ (Ward et al., 2002, Stem Cells. 20:472-475) and Lipofectamine 2000™ (Rui et al., 2006, Theriogenology. 65(4):713-720), is also broadly used. Although the liposome-mediated method has a high transfection efficiency, it is still unclear if stem cells treated with cationic lipids maintain pluripotency (Ma et al., 2004, Methods. 33(2):113-120).

Recently, Amaxa™ nucleofector, an electroporation-based method was developed to deliver plasmid DNA directly into the nucleus of murine embryonic cells (Lakshmipathy et al., 2004, Stem Cells. 22(4):531-543; Sieme et al., 2005, Stem Cells Dev. 14(4):378-383). Although the method has a high transfection efficiency, a strong decrease of expression level of the exogenous gene was observed within the first week, and a complete loss of the expression was observed in nearly half of the proliferation of the stem cells. As a result, nucleofection technique seems to be an unstable transfection method for stem cells (Lorenz et al., 2004, Biotechnol. Lett. 26:1589-1592). Furthermore, human embryonic stem cells are quite different from murine embryonic cells, especially gene expression and regulation of neural differentiation. The model established with murine embryonic cells cannot be used directly for human embryonic cells (Przyborski et al., 2003, Stem Cells 21:459-471).

Therefore, it is necessary to develop a method for introducing an exogenous gene into a stem cell that expresses stably and remains pluripotency.

### SUMMARY OF THE INVENTION

The invention relates to a method for introducing an exogenous gene into a stem cell. The method comprises a specific condition for electroporation. The introduced exogenous gene is expressed stably and the stem cell still retains pluripotency.

One subject of the invention is to provide a method for introducing an exogenous gene into a stem cell comprising introducing the exogenous gene into the stem cell using electroporation; wherein the electroporation is performed by applying 1 to 4 electric pulses, and each electric pulse is generated at a voltage in the range from about 100 V/cm to about 300 V/cm for a period from about 5 ms to about 30 ms.

Another object of the invention is to provide a stem cell stably expressing an exogenous gene, wherein the exogenous gene is introduced by using the method as described above.

Still another object of the invention is to provide a transgenic animal comprising the stem cell as described above.

Still another object of the invention is to provide a transgenic animal, which is regenerated from the stem cell as described above.

Still another object of the invention is to provide a pharmaceutical composition comprising the stem cell as described above.

### BRIEF DESCRIPTION OF THE DRAWINGS

FIG. 1 shows microscopic view of passage 0 and 1 of the pES/GFP⁺ cell line expressing pAAV-hrGFP.

FIG. 2 shows microscopic view of the pES/GFP⁺ cell line expressing pAAV-hrGFP subcultured for 1 to 4 days.

FIG. 3 shows microscopic view of three strains of the pES/GFP⁺ cell line expressing pAAV-hrGFP.

FIG. 4 shows microscopic view of the pES/GFP⁺ cell line expressing pAAV-hrGFP after freezing and thawing.

FIG. 5 shows microscopic view of the pES/GFP⁺ cell line expressing pAAV-hrGFP that highly expresses Oct-4, AP, SSEA-4, TRA-1-60 and TRA-1-81 under immunocytochemistry staining.

FIG. 6 shows microscopic view of chromosome karyotype of the pES/GFP⁺ cell line expressing pAAV-hrGFP.

FIG. 7 shows microscopic view of embryoid bodies of the pES/GFP⁺ cell line expressing pAAV-hrGFP.

FIG. 8 shows microscopic view of the pES/GFP⁺ cell line expressing pAAV-hrGFP that express nestin (neural precursor marker) and NF68 (neuronal marker) by immunocytochemistry staining.

FIG. 9 shows microscopic view of neural network differentiated from the pES/GFP⁺ cell line expressing pAAV-hrGFP.

### DETAILED DESCRIPTION OF THE INVENTION

The aim of the invention is to provide a method for introducing an exogenous gene into a stem cell comprising introducing the exogenous gene into the stem cell using electroporation; wherein the electroporation is performed by applying 1 to 4 electric pulses, and each electric pulse is generated at a voltage in the range from about 100 V/cm to about 300 V/cm for a period from about 5 ms to about 30 ms.

As mentioned above, the term “a stem cell” refers to a cell that has the property of prolongated proliferation and is able to differentiate into an adult cell having specific morphology and physiological functions. Preferably, the stem cell is derived from a mammal; more preferably, the mammal is selected from the group consisting of human, monkey, mouse, pig, and cattle; most preferably, the mammal animal is a human or pig. Pigs are similar to human beings in anatomy, immunology and physiology, and the organ, size of pigs is also similar to that of human beings. Therefore, pigs are regarded as an excellent animal model for biomedical study of human (Prellea et al., 1999, Cells Tissues Organs 165:220-236). Moreover, the properties of porcine embryonic stem cell are similar to those of human embryonic stem cell. Both stem cells need to be co-cultured with feeder cells. In morphology and cell biology, porcine and human embryonic stem cells both form embryonic stem cell colonies that are a specific morphology of stem cells, and they both express stage-specific embryonic antigens 3 and 4 (SSEA 3/4). On the other hand, murine embryonic stem cell specific antigen, SSEA 1, is not expressed in both porcine and human embryonic stem cell. Conclusively, the method and condition for the porcine stem cell are also applicable in the human stem cell.

According to the resource, the stem cell according to the invention is preferably embryonic stem cell, embryonic germ cell or adult stem cell; wherein the embryonic stem cell is derived from the inner cell mass of preimplantation blastocyst; the embryonic germ cell is derived from the primordial germ cells of the primitive genital ridge in the fetus; the adult stem cell is derived from an adult tissue of bone marrow, blood, sclera, omentum, hippocampus, olfactory bulb, skeletal muscle, gums, liver, derma, inner epithelial of intestine, or pancreas.

As used herein, the term “an exogenous gene” refers to a gene or gene fragment that is not derived from the stem cell itself. The exogenous gene according to the invention is chosen depending on the purpose. In one embodiment of the invention, the exogenous gene is a reporter gene. Preferably, the reporter gene is a green fluorescent protein (GFP) gene. The reporter gene can be used with other exogenous genes. By observing the express of the reporter gene, introducing and expressing of the exogenous gene co-introduced can be evaluated.

For convenient manipulation of electroporation, the exogenous gene according to the invention is preferably contained in a vector; preferably, the vector is derived from a virus. In one more preferred embodiment of the invention, the virus is a retrovirus or an adeno-associated virus type-2 (AAV-2).

As used herein, the term “electroporation” refers to a process of introducing an exogenous gene into a target tissue or organ with an electronic field. When applying the direct electronic field, a micro channel sized about 105 to 115 micrometers is formed on the cell membrane surface. Such channel opens for several mini seconds to seconds and re-closed automatically. During the period, biological macromolecule such as DNA is able to enter a cell through the micro channel. Electroporation introduces the exogenous gene efficiently and can be applied in several tissues and organs with a higher transfection efficiency. According to the invention, the voltage is in a range from about 100 V/cm to about 300 V/cm; preferably from about 100 V/cm to about 150 V/cm. The period is from about 5 ms to about 30 ms; preferably from about 5 ms to about 10 ms. The electroporation is performed by applying 1 to 4 electric pulses; preferably 1 or 2 electric pulses. More preferably, the electroporation is performed in a condition selected from the group consisting of

2 electric pulses; and each electric pulse is generated at a voltage of 150 V/cm for a period of 10 ms;

2 electric pulses; and each electric pulse is generated at a voltage of 150 V/cm for a period of 20 ms; and

2 electric pulses; and each electric pulse is generated at a voltage of 250 V/cm for a period of 10 ms.

Most preferably, the electroporation is performed by applying 2 electric pulses; and each electric pulse is generated at a voltage of 150 V/cm for a period of about 10 ms. The success rate of introducing the exogenous gene with the method according to the invention reaches 30% and the survival rate of the stem cell reaches 90%. The introduced exogenous gene is able to express stably in the stem cell. After subcultured for over 50 passages in 12 months, the exogenous gene still expresses stably.

The invention also relates to a stem cell stably expressing an exogenous gene, wherein the exogenous gene is introduced by using the method as described above. In one embodiment of the invention, the stem cell is observed to express stem cell markers of Oct-4, AP, SSEA-4, TRA-1-60, and TRA-1-81. It is shown that the stem cell stably expressing an exogenous gene retains its pluripotency. In one embodiment of the invention, the stem cell is induced to differentiate to a neural cell. Preferably, the stem cell is a stem cell strain. As used herein, the term “a stem cell strain” refers to an inbred stem cell that maintain the parents' properties after subculture.

The invention also relates to a transgenic animal comprising the stem cell as described above. The stem cell according to the invention is able to introduced into an animal to obtain the transgenic animal.

The invention still also relates to a transgenic animal, which is regenerated from the stem cell as described above. The stem cell according to the invention has the pluripotency and is able to regenerate to the transgenic animal.

The invention still also relates a pharmaceutical composition comprising the stem cell as mentioned above. The stem cell according to the invention is able to differentiate into an adult cell for repairing adult cells, tissue or organs suffered from mechanical, chemical and biological damage and diseases.

The following Examples are given for the purposes of illustration only and are not intended to limit the scope of the present invention:

### EXAMPLE 1

### Culture of Porcine Embryonic Stem Cells

Porcine embryonic stem cells were derived and cultured as described previously (Chen et al., 1991, J. Chin. Soc. Anim. Sci. 20:326-339; Chen et al., 1999, Theriogenology 52:195-212). Briefly, the pluripotent porcine embryonic stem cells were propagated on a feeder layer of mitomycin C (Sigma®, M-0503) inactived STO cells. STO cells were available from American Type Cluture Collection (ATCC; http://www.atcc.org; Cat. #CRL-1503, USA). The cells were cultured in ESM containing Dulbecco's modified Eagle's medium (DMEM; Gibco®, 11965-092) supplemented with 10% fetal bovine serum (Gibco®, 16000-044), 0.1 mM β-2-mercaptoethanol (Sigma® M-7522), 1% MEM non-essential amino acids (Bio West®, X-0557), 1 mM L-glutamine (Bio West®, X-0550) at 37° C. with an atmosphere of 5% CO₂ in air. For the maintenance of undifferentiation, the porcine embryonic stem cells were subcultured about once a week through 0.25% trypsin-EDTA (Gibco®, 25200-072) digesting and the porcine embryonic stem colonies were transferred onto a freshly mitomycin C inactived STO feeder layer.

### EXAMPLE 2

### Electroporation

The porcine embryonic stem cells were washed once with phosphate-buffered saline (PBS; Gibco®, 14190-136) added with 0.25% trypsin/EDTA and incubated at 37° C. and 5% CO₂ for 5 minutes for digesting the embryonic stem cell colonies and obtaining single embryonic stem cell suspension. The cell suspension was pooled and span down for 5 minutes at 1,000 rpm, and the pellet was washed twice with 5 ml PBS for removing serum. The cells were adjusted to approximately 5×10⁴ cells for each electroporation.

Approximately 20 μg of pAAV-hrGFP Control Plasmid (Stratagene®, 240074) containing green fluoresce protein gene was used as an exogenous gene. The prepared single embryonic stem cells were incubated with pAAV-hrGFP Control Plasmid in an electroporation cuvette (Cuvettes Plus™, Model No. 620, BTX, San Diego, Calif., USA) at 4° C. ice for 5 minutes. The Electro Cell Manipulator (BTX® ECM 2001, San Diego, Calif., USA) was used for electroporation. The conditions were listed in Table 1.

TABLE 1

| Condition No. | Voltage (V/cm) | Duration (ms) | Pulse No. (times) |
|---|---|---|---|
| 1 | 150 | 10 | 2 |
| 2 | 150 | 20 | 2 |
| 3 | 250 | 10 | 2 |
| 4 | 100 | 10 | 2 |
| 5 | 130 | 10 | 2 |
| 6 | 150 | 10 | 2 |
| 7 | 180 | 10 | 2 |
| 8 | 200 | 10 | 2 |
| 9 | 230 | 10 | 2 |
| 10 | 250 | 10 | 2 |
| 11 | 300 | 10 | 2 |
| 12 | 150 | 10 | 1 |
| 13 | 150 | 10 | 2 |
| 14 | 150 | 10 | 3 |
| 15 | 150 | 10 | 4 |
| 16 | 150 | 5 | 2 |
| 17 | 150 | 10 | 2 |
| 18 | 150 | 20 | 2 |
| 19 | 150 | 30 | 2 |

After electroporation, the embryonic stem cells were incubated at room temperature for 5 minutes and the cuvette was then rinsed twice with ESM to recover all the electroporated cells. The cells were placed onto a fresh mitomycin C inactived STO feeder layer.

### EXAMPLE 3

### Selection and Subculture

Five or six days after electroporation, small embryonic stem cell colonies became visible in a 4-well dish. The embryonic stem colonies expressing green fluorescence protein were picked up by mechanical selection in a laminar flow hood. The picked colonies were treated with 50 μl 0.25% trypsin/EDTA for 5 minutes at 37° C. and 5% CO₂. After being trypsinized, the embryonic stem cell colonies were pipetted up and down gently to dissect the colonies into some small clumps. The embryonic stem cell clumps were placed onto a fresh mitomycin C inactived STO feeder layer.

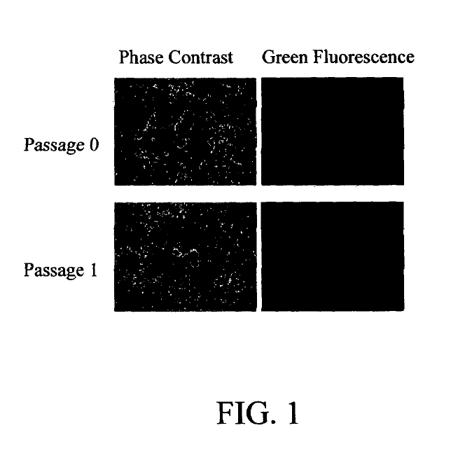

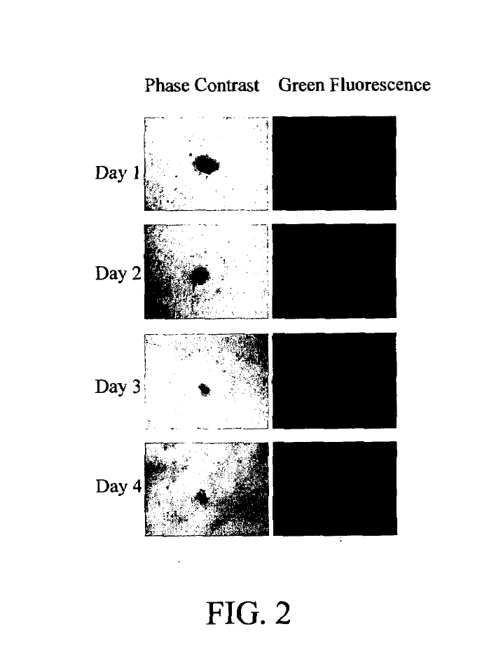

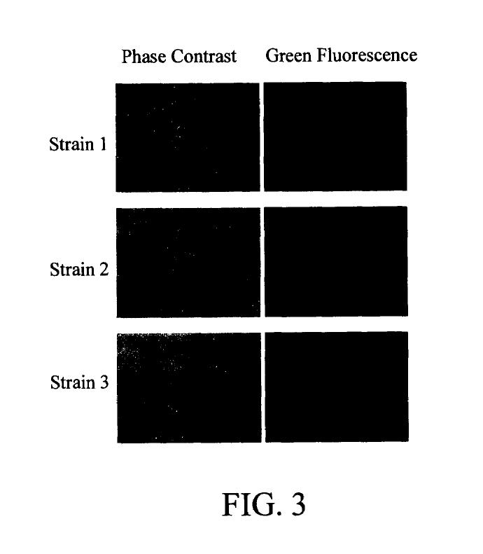

According to Condition 1, 14 tests were performed and obtained 30 pES/GFP⁺ colonies. Because the exogenous gene entered the genome of the stem cell randomly, some pES/GFP⁺ colonies failed to express pAAV-GFP stably. After 3 to 4 passages, the green fluorescence protein expression was lost. Finally, three embryonic stem cell strains stably expressing green fluorescence protein were selected (referring to FIGS. 1 to 3). The three strains were subcultured for more than 50 passages in 12 months.

The result of Conditions 4 to 19 are shown in Table 2.

TABLE 2

| Condition No. | Transfection rate (%) | Survival rate (%) |
|---|---|---|
| 4 | 5 | 90 |
| 5 | 10 | 90 |
| 6 | 30 | 90 |
| 7 | 10 | 75 |
| 8 | 5 | 75 |
| 9 | 0 | 60 |
| 10 | 0 | 60 |
| 11 | 0 | 50 |
| 12 | 10 | 90 |
| 13 | 30 | 90 |
| 14 | 10 | 70 |
| 15 | 0 | 20 |
| 16 | 10 | 80 |
| 17 | 30 | 90 |
| 18 | 10 | 60 |
| 19 | 0 | 60 |

### EXAMPLE 4

### Freezing and Thawing

For cryopreservation, approximately 20 small embryonic stem cell clumps were cryopreservated in 50 μl freezing medium in an 1 ml CryoTube ™ Vials (Nunc®, 377267, Denmark). The freezing medium contained 10% dimethylsulfoxide (DMSO; Sigma®, D-5879) and 90% fetal bovine serum. The embryonic stem cell clumps in the freezing medium were immediately moved to −20° C. for 4 hours, and then moved to −80° C. overnight and −196° C. liquid nitrogen for a long-period cryopreservation. To thaw the embryonic stem cells, the cells were thawed at 37° C. in a water bath and rinsed the cryotube twice with 100 μl ESM. The thawed embryonic stem cell clumps were placed onto a fresh mitomycin C inactived STO feeder layer and the DMSO was removed from the freezing medium in the second day. The embryonic stem cell colonies became apparent in 2 to 3 days and were ready for subculture in 5-7 days.

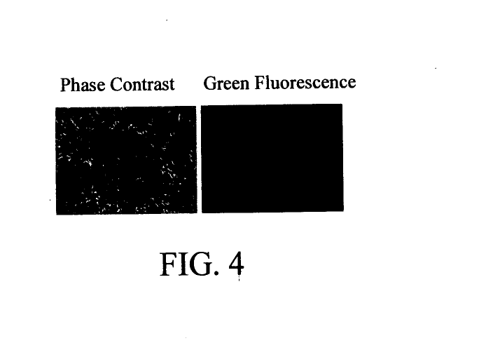

The result is shown in FIG. 4.

### EXAMPLE 5

### Pluripotency Analysis of Embryonic Stem Cells

To assay the pluripotency of the transfected porcine embryonic stem cells, immunocytochemistry (ICC) was used to analyze the expression of cell surface markers. The embryonic stem cell colonies were fixed with 10% formalin for 30 minutes and then washed with PBS and permeabilized with 0.1% Triton X-100 for 10 minutes, and incubated in 5% FBS blocking buffer for 2 hours. After blocking, the colonies were washed with PBS and then incubated with primary antibodies at 4° C. overnight. The undifferentiated pluripotent marker antibodies and the dilutions were utilized as follows: octamer-binding transcription factor (Oct-4; 1:200, Chemicong, AB3209), alkaline phosphatase (AP; 1:200, Chemicon®, MAB4349), stage specific embryonic antigen-3 (SSEA-3; 1:200, Chemicon®, MAB4303), stage specific embryonic antigen-4 (SSEA-4; 1:200, Chemicon®, MAB4304), tumor related antigen-1-60 (TRA-1-60; 1:200, Chemicon, MAB4360) and tumor related antigen-1-81 (TRA-1-81; 1:200, Chemicon®, MAB4381). The colonies were washed with PBS and treated with secondary antibodies that were either Rhodamine (TRITC)-conjugated AffiniPure Rabbit Anti-Mouse IgG, IgM or Goat Anti-Rat IgG (Jackson Immuno Research® 315-025-003, 315-025-044, 112-025-003, 1:200) for 20 minutes. Finally, the colonies were washed with PBS and incubated in PBS for assaying with Leica® DM IRB fluorescent microscope.

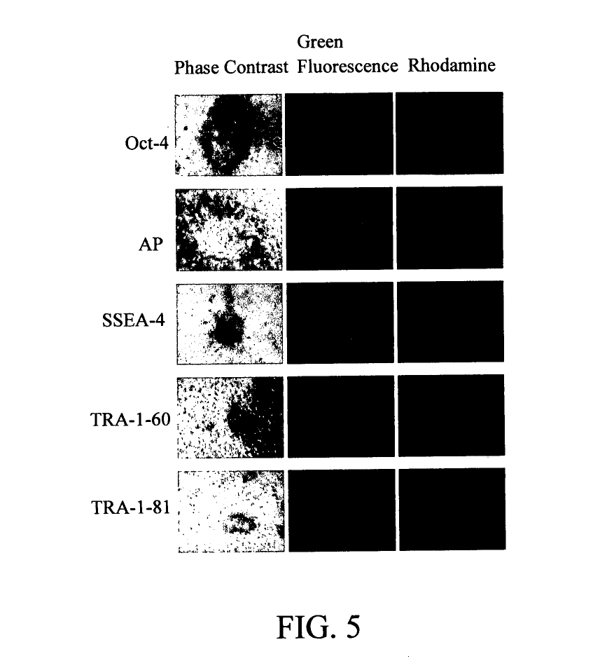

The results are shown in FIG. 5. It can be seen that the embryonic stem cells after electroporation highly express cell markers of Oct-4, AP, SSEA-4, TRA-1-60, and TRA-1-81. The transfected pAAV-hrGFP was proven to retain pluripotency of undifferentiated stem cells.

### EXAMPLE 6

### Chromosome Morphology Assay

The embryonic stem cells were cultured in a 3-cm Petri dish overnight. The cell cycle was then stopped by treating with Colcemid (KaryoMax® Colcemid solution, Gibco®, 15212-012). The cells were suspended in 0.56% KCl and fixed on a slide with methanol and glacial acetic acid (3:1). The cells were then dyed with 5% Gurr's Giemsa solution (Gibco®, 10092-013).

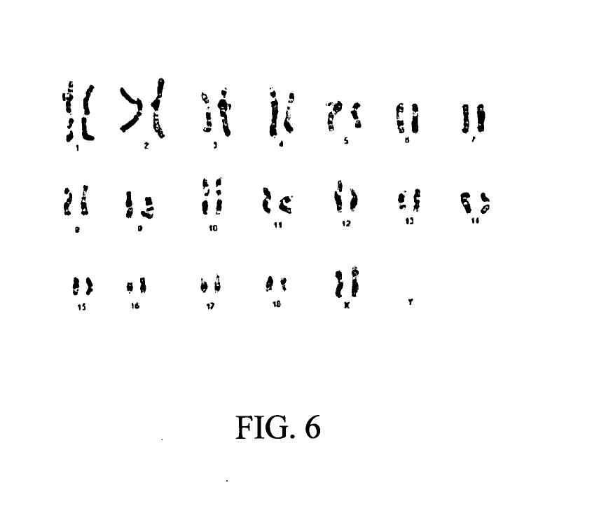

Referring to FIG. 6, it shows that pAAAV-hrGFP⁺ embryonic stem cell has a normal chromosome morphology of a female (36+XX).

### EXAMPLE 7

### Embryoid Body Formation Assay

The pES/GFP⁺ cells were cultured by the ‘hanging drop’ method (Wobus et al., 1997, J. Mol. Cell Cardiol. 29(6):1525-139) in a bacteriological Petri dish. The embryonic stem cells were shown to form embryoid bodies in vitro and undergo specific morphological changes (Rohwedel et al., 1999, Cells Tissues Organs 165:190-202).

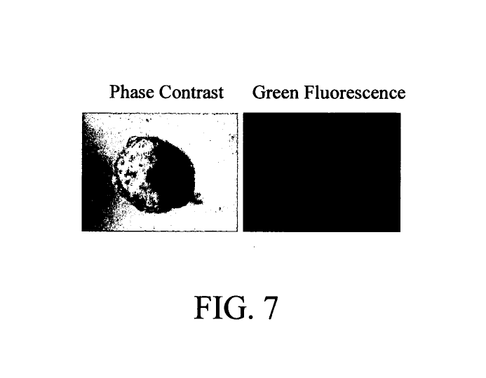

The result is shown in FIG. 7.

### EXAMPLE 8

### Differentiation of Neural Cells

Twelve days after embryoid body forming, the embryoid bodies were induced to differentiate into specialized neural cells with 1 μM retinoic acid (RA, Sigma®, R-2625). The medium contained DMEM/F12 (Sigma®, D-8437), 10% FBS (Gibco®, 16000-044), 20 ng/mL human recombinant epidermal growth factor, (hrEGF, Gibco®, 13247-051), 20 ng/mL human recombinant basic fibroblast growth factor (hrbFGF, Gibco®, 13256-029), 1:100 N2 supplement, Gibco®, 17502-048).

### EXAMPLE 9

### Immunocytochemical Staining

Twelve days after neural differentiation and immunocytochemical stained at day 4 (12/4), the neural-like cells were fixed in 10% formalin at room temperature for 30 minutes. The cells were then incubated with primary antibodies anti nestin (neural precursor marker, 1:200, Chemicon® AB5922) and neurofilament protein 68 kDa (NF68, neuronal marker, 1:200, Chemicon AB1983) at 4° C. overnight and subsequently incubated with the secondary antibody of Rhodamine (TRITC)-conjugated AffiniPure™ Goat Anti-Rabbit IgG (Jackson Immuno Research® 111-025-003, 1:200) for 20 minutes. Finally, the colonies were washed with PBS and incubated in PBS for assaying with Leica®R DM IRB fluorescent microscope.

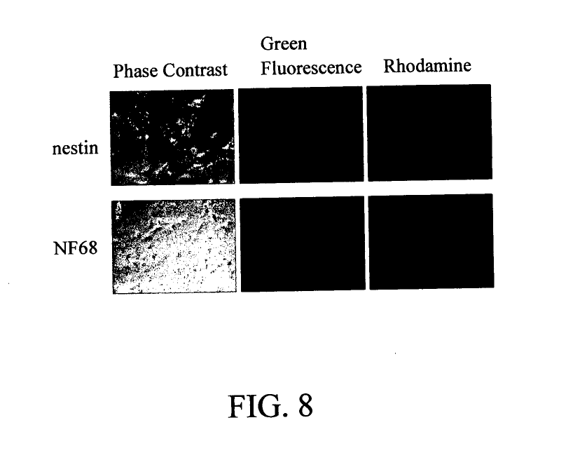

The result is shown in FIG. 8. It can be seen that the neural cells differentiated from embryonic stem cell express neural specific cell markers.

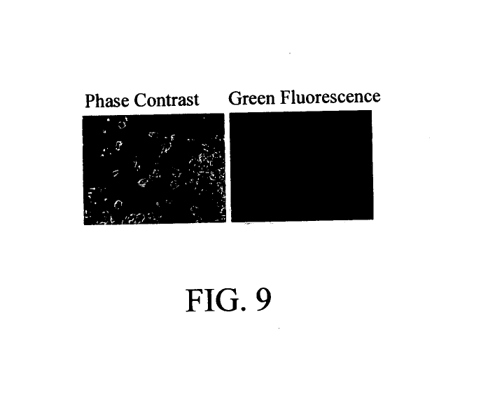

The cells were further cultured for 10 days (as shown in FIG. 9), a specific morphology of neural network was observed.

While embodiments of the present invention have been illustrated and described, various modifications and improvements can be made by persons skilled in the art. It is intended that the present invention is not limited to the particular forms as illustrated, and that all the modifications not departing from the spirit and scope of the present invention are within the scope as defined in the appended claims.

1. A method for introducing an exogenous gene into a stem cell comprising introducing the exogenous gene into the stem cell using electroporation; wherein the electroporation is performed by applying 1 to 4 electric pulses, and each electric pulse is generated at a voltage in the range from about 100 V/cm to about 300 V/cm for a period from about 5 ms to about 30 ms.

2. The method according to claim 1, wherein the stem cell is derived from a mammal.

3. The method according to claim 2, wherein the mammal is selected from the group consisting of human, monkey, mouse, pig, and cattle.

4. The method according to claim 2, wherein the mammal animal is a pig.

5. The method according to claim 2, wherein the mammal animal is a human.

6. The method according to claim 1, wherein the stem cell is an embryonic stem cell, an embryonic germ cell or an adult stem cell.

7. The method according to claim 6, wherein the embryonic stem cell is derived from an inner cell mass of preimplantation blastocyst.

8. The method according to claim 6, wherein the embryonic germ cell is a primordial germ cell derived from the primitive genital ridge.

9. The method according to claim 1, wherein the adult stem cell is derived from an adult tissue of bone marrow, blood, sclera, omentum, hippocampus, olfactory bulb, skeletal muscle, gums, liver, derma, inner epithelial of intestine, or pancreas.

10. The method according to claim 1, wherein the exogenous gene is contained in a vector.

11. The method according to claim 10, wherein the vector is derived from a virus.

12. The method according to claim 11, wherein the virus is a retrovirus or an adeno-associated virus type-2.

13. The method according to claim 1, wherein the exogenous gene is a reporter gene.

14. The method according to claim 13, wherein the reporter gene is a green fluorescent protein gene.

15. The method according to claim 1, wherein the voltage is in a range from about 100 V/cm to about 150 V/cm.

16. The method according to claim 1, wherein the period is from about 5 ms to about 10 ms.

17. The method according to claim 1, wherein the electroporation is performed by applying 1 or 2 electric pulses.

18. The method according to claim 1, wherein the electroporation is performed in a condition selected from the group consisting of
2 electric pulses; and each electric pulse is generated at a voltage of 150 V/cm for a period of 10 ms;
2 electric pulses; and each electric pulse is generated at a voltage of 150 V/cm for a period of 20 ms; and
2 electric pulses; and each electric pulse is generated at a voltage of 250 V/cm for a period of 10 ms.

19. The method according to claim 1, wherein the electroporation is performed by applying two electric pulses; and each electric pulse is generated at a voltage of 150 V/cm for a period of about 10 ms.

20. A stem cell stably expressing an exogenous gene, wherein the exogenous gene is introduced by using the method according to claim 1.

21. The stem cell according to claim 20, which is a stem cell line.

22. A transgenic animal comprising the stem cell according to claim 20.

23. A transgenic animal, which is regenerated from the stem cell according to claim 20.

24. A pharmaceutical composition comprising the stem cell according to claim 20.
